(19) United States
(12) Patent Application Publication
(10) Pub. No.: US 2006/0124130 A1
(43) Pub. Date: Jun. 15, 2006

(54) Ventilation system for respiratory devices

(76) Inventor: Jorge Bonassa, Sao Paulo (BR)

Correspondence Address: BROOKS KUSHMAN P.C., 1000 TOWN CENTER, TWENTY-SECOND FLOOR, SOUTHFIELD, MI, 48075, US

(21) Appl. No.: 10/559,946

(22) PCT Filed: Aug. 4, 2004
(86) PCT No.: PCT/BR04/00142

§ 371 Date: Dec. 8, 2005

(30) Foreign Application Priority Data
Dec. 26, 2003 (BR) PI0306282-1

(51) Int. Cl. A61M 16/00; A62B 7/00

(52) U.S. Cl. 128/204.28; 128/204.18

(57) ABSTRACT

The present invention refers to a ventilation system used in hospital respiratory devices for the administration of anesthesia to newly born, pediatric and adult patients. More specifically, the present invention refers to a ventilation system that promotes a respiratory circuit with re-inhalation for the administration of anesthesia and that overcomes all inconveniences and deficiencies existing in respiratory devices of the state of the art. The ventilation system in respiratory devices comprises a bellows assembled within a reservoir, which is provided with a manifold with various gas exhaling valves, more specifically release valves to take out the excess of gases, and an exhaling valve for control gases. It is additionally constituted of a valve for the free flow of oxygen to quickly renew or replace the gases inside the respiratory circuit.

### FIELD OF THE INVENTION

The present invention refers to a ventilation system for hospital respiratory devices that are used to administer anesthesia in patients.

More specifically, the invention refers to a ventilation system used in a respiratory circuit with re-inhalation by means of an upstream bellows that is activated by a microprocessed electronic ventilator and provided with a fresh gas control system.

The present invention is appropriate for the use in respiratory devices to administer anesthesia in newly born, pediatric and adult patients.

Additionally, the ventilation system of the present invention controls appropriately, efficiently and safely the excess of gas inside the respiratory circuit, thus eliminating the waste of fresh gas, promoting more efficient and safe breathing to patients, and not requiring harmful efforts for the respiratory cycle.

### BACKGROUND OF THE INVENTION

The inhalatory administration of anesthesia is made by means of a breathing circuit that makes the partial re-inhalation of gases exhaled by patients. The purpose is to reduce the consumption of anesthesia, such as halogenated agents, because human organism absorbs only a small quantity of anesthesia at each respiratory cycle. Furthermore, said respiratory circuits have the purpose to reduce the environmental pollution caused by the exhaustion of said agents.

Ventilation systems for anesthesia that uses respiratory circuits with re-inhalation, are different from those used, for example, in intensive care units, in which the gas controlled by the ventilation system is the same gas inhaled by the patient during the inhalation stage, and the gas is exhaled to the environment at the exhalation stage. In cases which re-inhalation occurs, it is necessary to separate the gas inhaled by the patient from the control gas in order to avoid eventual contamination of the re-inhaled air.

Since the invention refers to a ventilation system for respiratory circuits with re-inhalation, the description below will focus more specifically on this kind of application. Therefore, as known by those skilled in the art, the gas contained in the respiratory circuit, which is inhaled by patients, is constituted by a portion of re-inhaled gas and by a portion of fresh gas continually introduced to the circuit.

The respiratory circuit is initially full filled with fresh gas and at that time the re-inhalation process starts with the reposition of a fraction of the gas contained in the circuit by continuously-feeding fresh gas. The concentration of this gas is controlled by a set of flowmeters that uses oxygen and nitrous oxide. The flowmeters are associated with a calibrated vaporizer that adjusts the concentration of anesthesia agents, such as isoflurane, sevoflurane, enflurane or desflurane.

Re-inhalation circuits used in the devices of the state of the art continuously feed fresh gas into the respiratory circuit. The fresh gas is collected in an expandable bag or bellows, depending on the selection of the type of ventilation system that may be manual, by means of the bag, or automatic, by means of a ventilator. Said selection is usually made by means of a manual/ventilator selecting valve.

In summary, in the manual ventilation system, the anesthesia physician presses the bag and, due the presence of two unidirectional valves, the gas is directed to the patient through the inhaling branch of the circuit and passes through a carbon dioxide (CO₂) absorber. When the anesthesia physician stops pressing the bag, the gas exhaled by the patient returns to the bag through the exhaling branch. Therefore, the unidirectional valves guides the direction of the flow during patient's inhalation and exhalation, forcing the gas to pass through the carbon dioxide (CO₂) absorber before patient's re-inhalation.

Usually, an adjustable pressure-limiting valve allows the release to the atmosphere of the excess of gas inside the circuit through an appropriate exhaustion system, since the circuit is continuously fed with fresh gas.

In the automatic ventilation system, the bellows is filled in and the respiratory circuit is pumped by the control gas introduced by the ventilator into a rigid reservoir, in which said bellows is assembled. Therefore, during exhalation, the ventilator depressurizes the internal area of the rigid reservoir through an exhaling valve, thus allowing the gas exhaled by the patient to accumulate inside the bellows. Usually, said bellows is placed to act upwardly, i.e. the filling in of the bellows lifts up its free extremity in order to overcome its own weight.

Since respiratory circuits with re-inhalation are continuously fed with fresh gas, the devices of the state of the art usually release the excess of gas in the respiratory circuit through a release valve, which is passive and placed in the bellows set. The purpose of this valve is to alleviate the excess of gas inside the respiratory circuit after the bellows is fully filled in, in order to avoid the excess of circuit pressurization above the previously set up pressure value.

These release valves as known in the state of the art represents a dead weight, which is enough to generate a pressure, around 3 to 5 hPa that is higher than the weight of the bellows, which statically weights about 2 to 3 hPa. Therefore, in the inhaling stage, the release channel is closed, usually by the action of a diaphragm, in order to isolate said release valve and to allow the pressurization of the respiratory circuit. However, at the start of the exhaling stage, when the release channel is already open, there is a pressure peak due the exhaling pressure peak and inertia of the bellows, thus making the pressure inside the bellows higher than the pressure achieved under static conditions. This overpressure is enough to open the release valve. Consequently, there is gas leakage before the bellows is fully filled in. This is very undesirable because inhibits the use of low fresh gas flow and requires the reposition of flow above the clinically desired level. There are many advantages in the use of low fresh gas flow in respiratory circuits to administer anesthesia, especially regarding safety, saving of costs, environmental and clinical aspects, as those skilled in the art can appreciate it.

Furthermore, the release systems of the state of the art present another inconvenience. There is dependence between the fresh gas flow and the residual pressure resulted in the respiratory circuit, i.e. the higher the fresh gas flow, the higher will be the residual pressure in the circuit. This makes the more difficult ventilator synchronism with the spontaneous breathing of the patient, consequently increasing the respiratory work and compromising hemodynamics, especially for cardiopathic patients.

The patents U.S. Pat. No. 5,398,675 and U.S. Pat. No. 5,507,280 disclose a release system activated by a shaft in contact with a flexible bag. However, this system is not appropriate for newly born and pediatric applications, since it promotes undesirable positive pressure during system operation. This inconvenience is caused by the fact that there is no constant relationship between the volume of the flexible reservoir and the position of its flexible wall, as well as between the sensor's contact area and consequently the force exerted by the bag and the required force to activate the release system.

The patent U.S. Pat. No. 5,678.540 discloses a system which purpose is to improve the pressure control inside the bellows and the reservoir, in order to allow the ventilation under controlled pressure. However, said system uses a conventional passive release valve of the state of the art, which is the same as those previously disclosed.

Another inconvenience disclosed by the devices of the state of the art relates to the valve for the free flow of oxygen, which purpose is to quickly replace or renew the gas inside the respiratory circuit. Usually, when manually pressed, said values control a free flow of 30 to 40 l/min of oxygen without anesthetic agent. However, in automatically configured respiratory circuits, said free flow of oxygen may cause excessive increase of volume and pressure sent to the patient and may cause serious damage to the respiratory system or even to patient's hemodynamic.

This kind of valve is observed in the patent U.S. Pat. No. 5,678,537, which discloses a system to avoid the increase of pressure in the respiratory circuit by means of an activated valve for the free flow of oxygen during the inhaling stage of the respiratory cycle. In this system, the respiratory cycle is interrupted whenever the valve for the free flow of oxygen is activated, opening the exhaling valve. However, despite eliminating the risk of pressure trauma caused by the overlaying of the respiratory cycle and the activation of the free flow of oxygen, this alternative interferes with the lung ventilation of the patient and may cause a reduction of oxygenation (hypoxia) and/or increase of CO₂ retention (hypercapnia), which may compromise the health of patients.

Therefore, it is verified that the ventilation systems of the state of the art have serious inconveniences to patients, especially because they do not meet the technical and medical requirements for the administration of anesthesia through respiratory circuits with re-inhalation, since they can generate inefficient and dangerous gas flow to patient's respiratory system.

### SUMMARY OF THE INVENTION

Therefore, it is an object of the present invention to provide a ventilation system for respiratory devices with re-inhalation, more specifically for the administration of anesthesia to newly born, pediatric and adult patients that overcomes all problems and inconveniences existing in the ventilation systems of respiratory devices of the state of the art.

Another object of the present invention is to provide a ventilation system for respiratory circuits with re-inhalation that promotes an effective control of the excess of fresh gas, as well as the control of the internal pressure of the respiratory circuit. The present invention can also adequate the respiratory circuit to the spontaneous breathing of patients, not requiring efforts from the respiratory system, besides providing an appropriate and controlled respiratory cycle to patients.

It is also a further object of the invention to provide a valve for the free flow of oxygen that actuates synchronously with the exhaling stage of respiratory cycles, in order to eliminate the risks of pressure trauma without intervening in the lung ventilation of patients.

The system of the present invention may be preferably used in a respiratory device to administer anesthesia with re-inhalation provided with a carbon dioxide (CO₂) absorbing system, which in turn is the object of another patent application filed by the applicant under the serial number BR PI 0305789-5, filed on Nov. 17, 2003.

### BRIEF DESCRIPTION OF THE DRAWINGS

These and other objects, enhancements and effects of the ventilation system object of this invention will be apparent for those skilled in the art from the detailed description below with relation to the attached figures that illustratively represent:

FIG. 1 illustrates a schematic diagram of the respiratory cycle with re-inhalation of a device with a ventilation system according to the present invention;

FIG. 2 illustrates a detailed sectional view of the bellows and reservoir set of the ventilation system according to the present invention;

FIG. 3 shows an alternative embodiment of the bellows and reservoir set according to the present invention, for the application in newly born treatments;

FIG. 4 shows a detailed sectional view of the manifold of the bellows and reservoir set according to the present the invention;

FIG. 5 shows a detailed sectional view of an alternative embodiment of the manifold according to the present invention; and

FIG. 6 shows a detailed sectional view of the valve for the free flow of oxygen according to the present invention.

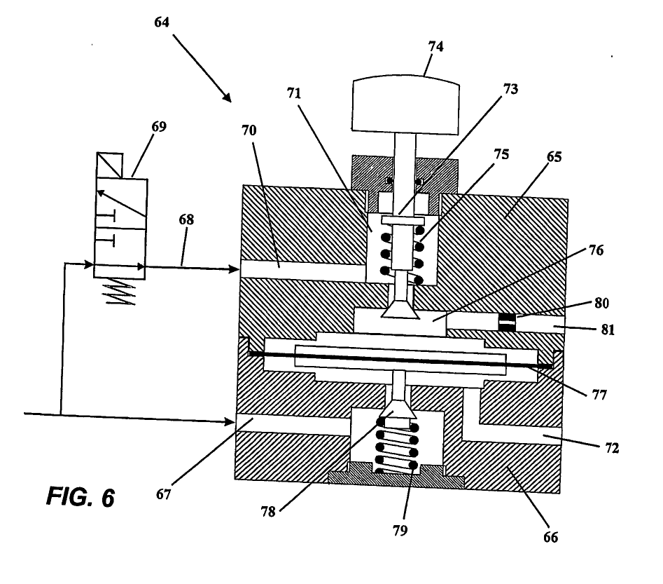

### DETAILED DESCRIPTION OF THE INVENTION

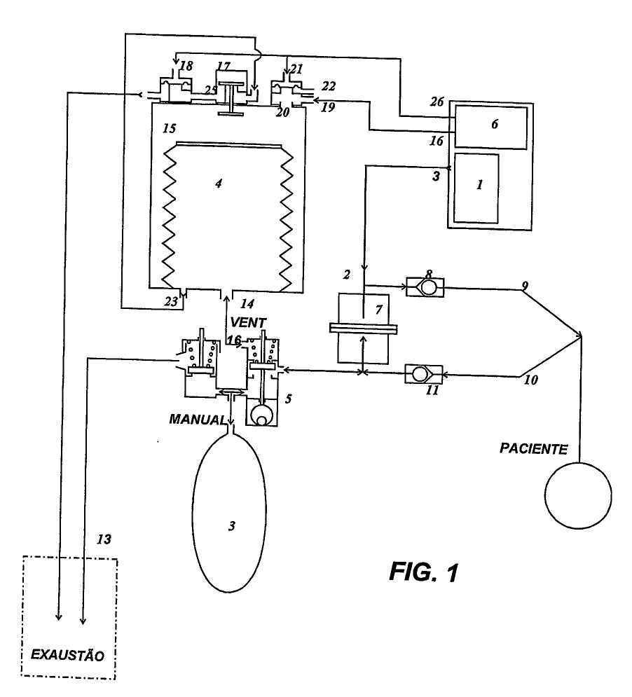

The FIG. 1, as mentioned above, shows a schematic diagram of the respiratory cycle with re-inhalation of a respiratory device provided with the ventilation system of the present invention. As illustrated in FIG. 1, the fresh gas coming from the anesthesia equipment (1), that adequately adjusts the composition of gases and the concentration of anesthetic agents, is introduced in the respiratory circuit through the fresh gas inlet (2). The gas is collected in a bag (3) or bellows (4), depending on the application requirements and according to the position of the selecting key (5), which determines the mode of operation of the device: manual or automatic through a lung ventilator (6).

In case the selecting key (5) is in the manual position, the bag (3) is filled in with gas, so that the anesthesia physician or any other specialist may manually pump the gas to patient. When the bag is pressed, the gas passes through a carbon dioxide absorber (7), through the inhaling unidirectional valve (8), through the inhaling tube (9), and insufflates the lung of the patient. When the anesthesia physician releases the bag (3), the gas exhaled by the patient passes through the exhaling tube (10), through the unidirectional exhaling valve (11), and returns to the bag (3). The purpose of the unidirectional valves (8) (11) is to force the passage of the gas flow through the carbon dioxide absorber (7) before the re-inhalation by the patient. For this case, an adjustable pressure limit valve (12) releases to the environment the excess of gas within the respiratory circuit by means of an appropriate exhaustion system (13), since the fresh gas is continuously fed within the circuit.

In case the selecting key (5) is in the ventilator/auto mode, the respiratory circuit works analogously to the manual mode. However, pumping is made by the bellows (4), which is filled in through the exhaling valve (21) by the route (14). Pumping is made through the lung ventilator (6) that pressurizes the internal side of the rigid reservoir (15), where said bellows (4) is assembled, through the inhaling route (16). In this case, the control of gas excess is made by a set of release valves, which comprises a release valve (17) activated by said bellows (4) and a release valve (18) activated by the lung ventilator (6) through the route (26). Said release valves (17), (18) are activated during the exhaling stage. This allows the excess of gas located inside the circuit to escape and to avoid circuit pressurization with values above the set up exhaling pressure value.

The inhaling stage of the respiratory circuit starts with the ventilator (6) sending inhaling flow through the routes (16), (19), and (20) into the rigid reservoir (15). At the same time, the exhaling valve (21) and the release valve (18) are closed in order to pressurize the internal side of the rigid reservoir (15) and to compress the bellows (4). Through the ventilation system according to the present invention, all parameters controlled by the lung ventilator (6), such as flow, pressure and volume, are fully transmitted to the gas located inside the bellows (4) and consequently transmitted to the gas flow inhaled by the patient. For these reasons, the ventilation system of this invention can be used in various modes of ventilation, including but not limited to Volume Control Ventilation—VCV, Pressure Control Ventilation—PCV, Pressure Support—PSV, VAPS, APRV, PAV, etc.

After the end of the inhaling stage, the exhaling stage starts. In this stage, the route (16) flow is closed, and the exhaling valve (21) and the release valve (18) are opened. The gas located inside the reservoir (15) is exhaled through the routes (20), (22), thus permitting the bellows (4) to expand up to the end of the exhaling stage of the patient. Said bellows (4) fully expands and activates the release valve (17), causing the exhaustion of the excess gas from the respiratory circuit through the routes (23), (24), (25) and through the release valve (18) activated by the ventilator (6).

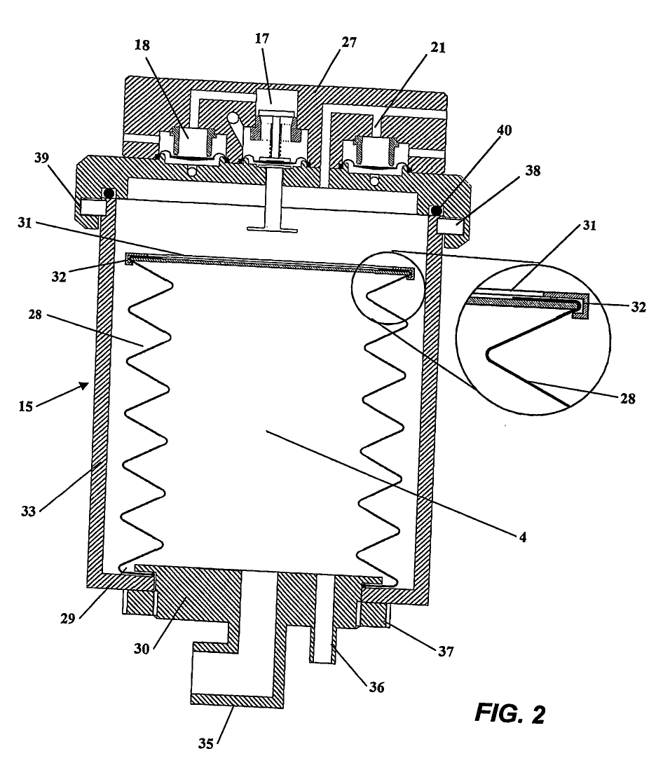

FIG. 2 illustrates the set constituted by the bellows (4) located upwardly within the reservoir (15) and by a manifold (27). Said bellows (4) is manufactured with flexible and sterilizable material, preferably silicone, which shape presents an accordion-like shape (28) to allow the expansion and contraction of the bellows (4) and thus offering low resistance and inertia. The base of said bellows (4) is provided with a ring-shaped opening (29) with circular cross section that is fitted in the base (30) of said rigid reservoir (15). The top of said bellows (4) is formed by a hard disk (31), preferably manufactured with aluminum with relatively small thickness, which is fitted under pressure through an external ring (32) in order to hermetically fix the accordion-like profile (28) to the hard disk (31), thus forming a flat and stable surface at the top of the bellows (4), as better observed in the detail of FIG. 2. Furthermore, said external ring (32) has the purpose to avoid eventual mixing between the control gas and the gas inhaled by the patient.

Therefore, the configuration of said bellows (4) permits the full transmission of all the pressure exerted over the external surface of the bellows to the gas of the respiratory circuit by the control gas inside the reservoir (15). Furthermore, it permits the inhaling effort exerted by the patient to be transmitted by the gas of the respiratory circuit to the control gas located inside the reservoir (15), thus not generating any resistance to the spontaneous breathing of the patient.

Said reservoir (15) comprises a main body (33), a base (30) and a manifold (27). Said main body (33) is manufactured with a transparent material, preferably polycarbonate or acrylics. Said base (30) is provided with a first connection (35) to couple the re-inhalation tube (not shown) and a second connection (36) which is connected to the manifold (27) in the release valve (17) for the exit of excess gases. Said base (30) is fitted in the lower portion of the main body (33) by means of a pressure screw (37). The edge of the circular opening (29) of said bellows (4) is hermetically pressed between said base (30) and the main body (33) to avoid the mixing between the respiratory circuit gas and the control gas.

The manifold (27) is assembled over the main body (33) by means of pins (38) located alongside the main body. The pins are fitted in indentations (39) located at the edge of said manifold (27), also provided with a sealing ring (40) to seal the main body (33) against the manifold (27).

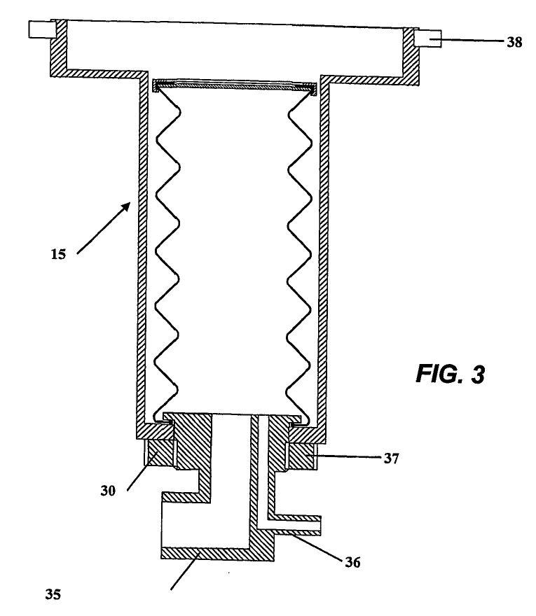

The volume capacity of the bellows (4) may vary according to the application. Bellows with varying capacity, for example from 250 to 1400 ml, are commonly used. For applications to newly born patients, the volume capacity should be small in order to minimize the total compressible volume of the respiratory circuit. FIG. 3 shows an alternative embodiment of the bellows (4) and reservoir (15) set, which presents a lower volume capacity than that shown in FIG. 2, but keeping the same principle of operation. However, said set uses the same manifold (27) used for reservoirs with a higher volume capacity, since the diameter of the upper end of the reservoir (X) is equal to the diameter of the reservoir used in the embodiment shown in FIG. 2.

The ventilation system of the invention may be used in various applications. No matter which is the volumetric capacity of the gases in the respiratory circuit, it is just required the substitution of the bellows/reservoir set.

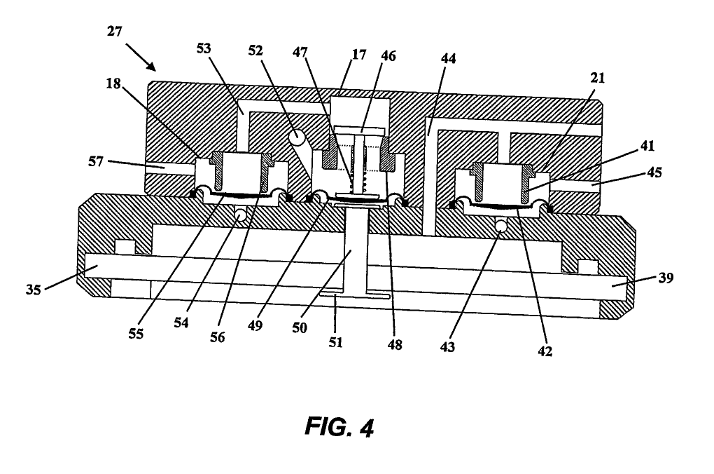

FIG. 4 shows in detail said manifold (27), preferably of aluminum, comprising the exhaling valve (21) and the release valves (17), (18) responsible for the control of gas excess in the respiratory circuit.

The exhaling valve of the control gas (21) comprises an air nozzle (41), which is opened and closed by means of the action of a flexible diaphragm (42), which in turn is activated by the pilot pressure through the gas inlet (43), with said pilot pressure being controlled by the lung ventilator (6) through a proportional solenoid valve and by an electronic control circuit provided with a pressure transducer, a microprocessor, and a PID control algorithm, such as those known by the man skilled in the art.

In the inhaling stage, the lung ventilator (6) pressurizes the internal side of the reservoir (15) through the channel (44) in order to control the pilot pressure in the gas inlet (43), thus closing the air nozzle (41) and consequently closing the exhaustion channel (45). In the exhaling stage, the pilot pressure at the gas inlet (43) will be reduced, thus permitting said flexible diaphragm (42) to open the air nozzle (41) and permitting the control gas to be exhausted through the channel (44), through the air nozzle (41), and through the channel (45). In this stage, it is possible to control the pilot pressure with the purpose to keep a positive pressure over the bellows (4), which is called PEEP (Positive End Expiratory Pressure).

The control of gas excess in the respiratory circuit is made by means of the release valves (17), (18). Each one of them is responsible for a control stage. More specifically, the first stage is performed by the valve (17), which is activated by the bellows (4) and comprises a cursor (46) which higher end is supported, under the action of a spring (47), over an air nozzle located within said manifold (27), and which lower end is supported over a flexible diaphragm (49). A second cursor (50) is assembled at the opposite side of the flexible diaphragm (49), which projects to the internal side of the reservoir (15) and has a disk (51) in its free end, which contacts the bellows (4).

Therefore, when the bellows (4) is fully filled in, the hard disk (31), located at the top of the bellows (4), touches the disk (51) of the cursor (50) and consequently activates the cursor (46) that opens the air nozzle (48), thus permitting the passage of the flow of gas excess from the inlet channel (52) to the outlet channel (53) and conducing the gas to the release valve (18) responsible for the second control stage. Said inlet channel (52) is connected to the outlet connection for gas excess (36) located at the base (30) of the reservoir (15). Furthermore, said diaphragm (49) safely separates the control gas located inside the reservoir (15) from the gas coming from the inlet channel (52), which is the excess of gas exhaled by the patient coming from the respiratory circuit. Therefore, the mixing between gases is inhibited and consequently a safe and healthy respiratory circuit is obtained.

The second stage is made by the release valve (18), similar to the exhaust valve of the control gas (21). The lung ventilator (6) also controls the release valve by the pilot pressure in the channel (54), which is equal to the pilot pressure of the channel (43). The release valve (18) is also provided with a flexible diaphragm (55) that closes and opens an air nozzle (56) according to the pilot pressure in the channel (54).

During the inhaling stage, the lung ventilator (6), through the channel (44), sends control gas flow into the reservoir (15) simultaneously closing air nozzles (41) and (56) through the flexible diaphragms (42), (55) and consequently pressurizing the reservoir (15), compressing said bellows (4) and the gas contained inside it. The gas is pumped to the patient, passing through the carbon dioxide absorbing system, through the inhaling unidirectional valve and insufflating patient's lung.

As previously explained, during the exhaling stage, the pilot pressure in the channel (43) is reduced, and consequently the pilot pressure of the channel (54) is also reduced. The pressure within diaphragms (42, 55) remain reduced, thus allowing the opening of the air nozzles (41, 56) and allowing the control gas exhalation through the channels (44, 55) and the interconnection of the channels (53, 57). However, the first stage to control the excess of gas remains closed due the force of the spring (47) that acts over the cursor (46) of the release valve (17). The air nozzle (48) remains closed and inhibits the exhaustion of the excess of gases through the channels (53), (57). Therefore, only when the bellows (4) is fully filled in, the hard disk (31) located on its top will activate the cursor (50), which will move the cursor (46) to open the air nozzle (48) to allow the passage of the excess of gas between the first stage and the second stage through the channels (53, 57).

The valve (18) of the second stage is controlled by the same pilot pressure of the exhaustion valve for the control gas (21). For this reason, the exhaustion pressure of the control gases and consequently the pressure inside the bellows is the same pressure kept by the ventilator inside the reservoir. This system allows the set up exhaling pressure value to be kept no matter which is the supplied fresh gas flow value, thus keeping the ventilation base line and allowing the spontaneous breathing of the patient with no additional effort to balance an eventual intrinsic PEEP.

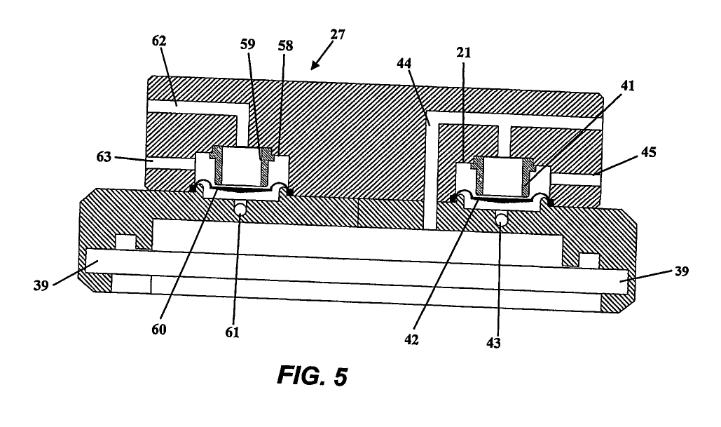

In an alternative embodiment of the manifold (27), as shown in FIG. 5, the control of gas excess is made by one stage. The control is formed by the release valve (58), which comprises an air nozzle (59) supporting a flexible diaphragm (60) activated by the pilot pressure through the channel (61), no matter which is the pilot pressure supplied through the channel (43) of the control gas exhaustion valve (21). In this case, the pilot pressure in the channel (61) is controlled by means of a second proportional solenoid that keeps the channel (62) closed during the inhaling stage. During the exhaling stage and only after the patient fully exhales, the air nozzle (59) is opened to allow the flow of gas excess to pass through the exhaustion channel (63).

The release valve (58) opens in a proportionally manner depending on the monitoring of the internal pressure increase of the respiratory circuit. The monitoring in turn is made by means of a pressure transducer, since it is required to keep the pressure in the circuit at the same value of the PEEP set up exhaling pressure value, in order to balance the pressure exerted by the weight of said bellows (4) itself. The full exhalation by the patient can be monitored, for example, by means of a specific device, such as a pneumotacograph located at the “Y” connection of the patient or even in the exhaling route.

Therefore, the release system for the excess of gases in the respiratory circuit of the invention solves the problems described in the state of the art, thus eliminating the risk of gas escape during the start of exhalation, besides keeping minimum residual pressure, of about 1 hPa, even by using high flow of fresh gas.

Additionally, the ventilation system of the present invention comprises a valve for the free flow of oxygen (64) provide with a solenoid valve (69) which is activated by the lung ventilator (6) synchronously with the inhaling stage of the respiratory cycle of the patient. The purpose of this valve (64) is quickly renewing the gases inside the respiratory circuit.

The valve for the free flow of oxygen (64) is constituted by two stages, a pilot (65) and a main one (66). Oxygen is fed through the channel (67) to the main stage and through the channel (68) by the “usually open” route of the solenoid valve (69), which is itself connected to the pilot stage through the channel (90) which in turn is interconnected to the manual activating valve (71).

The oxygen flow to the respiratory circuit is released through the channel (72) and occurs simultaneously when the solenoid (69) is turned off and the cursor (73) is manually activated by the key (74), therefore overcoming the spring (75) pressure and consequently allowing the oxygen flow between the channel (70) and the chamber (76). Hence, the activation of the pressure of the pilot stage over the diaphragm (77) causes the movement of the cursor (78), overcoming the force of the spring (79) and interconnecting the inlet (67) and outlet (72) channels of the main stage.

The manual valve (71) is closed by the action of the spring (75) over the cursor (73) and by the depressurization of the chamber (76), which occurs through the restrictor (80) located in the channel (81), thus closing the main stage due to the action of the spring (79) under the cursor (78). The solenoid (69) is activated by the lung ventilator (6) synchronously with the inhaling stage of the respiratory cycle of the patient, i.e. during the inhaling stage. The feeding of oxygen through the channel (70) is interrupted and consequently the pilot pressure over the diaphragm (77) and the flow of oxygen through the outlet channel (72) is interrupted, even if the pilot stage had been manually activated.

The valve for the free flow of oxygen of the present invention remains in operation even in the lack of power supply, thus allowing its operation for example by means of manual ventilation. This promotes a better safety during the administration of anesthesia, since it is possible to work even in case of lack of power supply or failure in the electronic system of the equipment. The synchronization of the free flow of oxygen during the exhaling stage avoids the risks of the equipments of the state of the art, allowing the operator to activate the flow at any moment of the ventilation, without the need to change the controlled standards, such as the respiratory frequency, and without need to interrupt the ventilation.

1. A ventilation system for respiratory devices, which comprises a bellows disposed inside a recipient provided with a manifold associated to a valve for the free flow of oxygen for the renewal of gases inside a re-inhalation respiratory circuit.

2. The ventilation system for respiratory devices of claim 1, wherein said bellows (4) is assembled upwardly inside the recipient (15).

3. The ventilation system for respiratory devices of claim 1, wherein said recipient (15) is formed by a main body (33), which upper end is closed by the said manifold (27) and the lower end is closed by a base (30).

4. The ventilation system for respiratory devices of claim 1, wherein said bellows (4) has an accordion-like profile (28), a base of which is provided with a circular opening (29) that fits in said base (30) of said reservoir (15), and said base is fixed by means of a pressure screw (34) on the main body (33).

5. The ventilation system for respiratory devices of claim 4, wherein the top of said bellows (4) is composed by a hard disk (31) that is fitted in said accordion-like profile (28) under pressure by means of an external ring (32).

6. The ventilation system for respiratory devices of claim 3, wherein the said base (30) of the recipient (15) also has a first connection (35) to couple a re-inhalation tube and a second connection (36) connected to said manifold (27).

7. The ventilation system for respiratory devices of claim 1, wherein said manifold (14) is provided with an exhaling valve (21) and at least with a release valve (17, 18, 58).

8. The ventilation system for respiratory devices of claim 1, wherein said valve for the free flow of oxygen (64) comprises two stages, a pilot stage (65) and a main stage (66).

9. The ventilation system for respiratory devices of claim 8, wherein the said pilot stage (65) is formed by a solenoid (69) connected to an inlet channel (70) of a manual activation valve (71), which is constituted by a cursor (73) supported over a spring (75) which is activated by a manual key (74).

10. The ventilation system for respiratory devices of claim 8, wherein said main stage (66) is formed by an oxygen inlet channel (67) and by a second cursor (78) supported over a spring (79), the second cursor and the spring being activated by the movement of a diaphragm (77) by means of pressure in a chamber (76) that separates both stages.

11. The ventilation system for respiratory devices of claim 1, wherein said bellows (4) is of a flexible and sterilizable material.

12. The ventilation system for respiratory devices of claim 5, wherein said hard disk (31) located on the top of the bellows (4) comprises aluminum.

13. The ventilation system for respiratory devices of claim 3, wherein said main body (33) of the recipient (15) is made of transparent material.

14. The ventilation system for respiratory devices of claim 3, wherein the edge of said manifold (27) is provided with indentations (39) that fit in pins (38) located at the upper portion of the main body (33).

15. The ventilation system for respiratory devices of claim 7, wherein said exhaling valve (21) and the release valve (18) located in said manifold (27) comprise an air nozzle (41, 56) provided with a flexible diaphragm (42, 55) activated by the pressure of an air inlet channel (43, 54).

16. The ventilation system for respiratory devices of claim 7, wherein said release valve (17) activated by the bellows (4) comprises a cursor (46), the upper end of which is supported on a spring (47) over an air nozzle located within said manifold (27), and the lower end of which is supported on a flexible diaphragm (49) which is provided at its opposite side with a second cursor (50) disposed at the end of a disk (51) that projects to the internal side of the reservoir (15).

17. The ventilation system for respiratory devices of claim 7, wherein the release valve (58) comprises an air nozzle (59) which supports a flexible diaphragm (60) activated by the pressure of a channel (61).

18. (canceled)

19. The ventilation system for respiratory devices of claim 10, wherein the diaphragm comprises silicone.

20. The ventilation system for respiratory devices of claim 11, wherein said bellows comprises silicone.

21. The ventilation system for respiratory devices of claim 13, wherein the recipient comprises acrylics.

22. The ventilation system for respiratory devices of claim 21, wherein the recipient comprises polycarbonate.

23. The ventilation system for respiratory devices of claim 15, wherein the flexible diaphragm comprises silicone.

24. The ventilation system for respiratory devices of claim 16, wherein the flexible diaphragm comprises silicone.

25. The ventilation system for respiratory devices of claim 17, wherein the flexible diaphragm comprises silicone.
